(19) United States
(12) Patent Application Publication
(10) Pub. No.: US 2023/0031270 A1
(43) Pub. Date: Feb. 2, 2023

(54) ELECTROMAGNETIC ACOUSTIC PROBE

(71) Applicant: ECOLE DE TECHNOLOGIE SUPERIEURE, Montréal (CA)

(72) Inventors: Pierre BELANGER, Montreal (CA); Aurélien THON, Montreal (CA)

(21) Appl. No.: 17/874,474

(22) Filed: Jul. 27, 2022

Related U.S. Application Data
(60) Provisional application No. 63/203,550, filed on Jul. 27, 2021.

(51) Int. Cl. G01N 29/24; G01N 29/04; G01N 33/2045

(52) U.S. Cl. CPC G01N 29/2493; G01N 29/2462; G01N 29/041; G01N 33/2045; G01N 2291/0289; G01N 2291/0234; G01N 2291/0423

(57) ABSTRACT

There is described a probe for non-destructive testing of a curved object, the probe comprising an arrangement of magnets and coils configured for generating shear horizontal guided waves for propagating longitudinally in the object, the probe having a top surface, a bottom surface, and two opposed ends extending between the top surface and the bottom surface, the bottom surface having a non-zero curvature between the two opposed ends and matable with an outer surface of the curved object.

### CROSS-REFERENCE TO RELATED APPLICATIONS

The present application claims the benefit of U.S. Provisional Patent Application No. 63/203,550 filed on Jul. 27, 2021, the contents of which are hereby incorporated by reference in their entirety.

### TECHNICAL FIELD

The application relates generally to measurement devices and, more particularly, to measurement devices that use ultrasonic guided waves for non-destructive testing.

### BACKGROUND OF THE ART

The inspection of corroded structures is crucial across many industries. Affected areas are often difficult to access due to other impeding structures, such as pipe support or insulation. This makes standard thickness gauging methods such as point-by-point ultrasonic thickness gauging impossible.

The use of ultrasonic guided waves in nondestructive testing enables rapid inspections over long distances. In a pipe, several modes can propagate, such as flexural (axisymmetric and non-axisymmetric) and torsional modes (axisymmetric and non-axisymmetric). Various techniques are known to propagate waves in pipes and detect defects. While these techniques are suitable for their purposes, improvements are desired.

### SUMMARY

In one aspect, there is provided a probe for non-destructive testing of a curved object, the probe comprising an arrangement of magnets and coils configured for generating shear horizontal guided waves for propagating longitudinally in the object, the probe having a top surface, a bottom surface, and two opposed ends extending between the top surface and the bottom surface, the bottom surface having a non-zero curvature between the two opposed ends and matable with an outer surface of the curved object.

In another aspect, there is provided a measurement system comprising at least one probe, a signal generating circuit for generating an emission signal, and a signal receiving circuit for receiving a detection signal. The probe comprises an arrangement of magnets and coils configured for generating shear horizontal guided waves for propagating longitudinally in the object, the probe having a top surface, a bottom surface, and two opposed ends extending between the top surface and the bottom surface, the bottom surface having a non-zero curvature between the two opposed ends and matable with an outer surface of the curved object.

### DESCRIPTION OF THE DRAWINGS

Reference is now made to the accompanying figures in which:

FIGS. 1A-1D are cross-sectional views of a probe arranged on a curved object for inspection thereof;

FIGS. 2A-2B are example configurations of a magnet array for a probe having a curved undersurface;

FIG. 3 is an example configuration of a coil for a probe having a curved undersurface;

FIG. 4A shows excitation energy obtained with a 20 magnets array when the excitation signal is a 20 cycle Hann windowed toneburst centered at 500 kHz;

FIG. 4B shows the phase velocity curves in a 10.2 mm thick steel plate;

FIGS. 5A-5B are block diagrams of example measurement systems;

FIGS. 6A-6D illustrate a maximum amplitude of Lorentz Forces generated on a steel pipe with a diameter of 323.8 mm and a thickness of 10.2 mm by probes having different configurations;

FIGS. 7A-7D illustrate an ultrasonic field generated on a steel pipe with a diameter of 323.8 mm and a thickness of 10.2 mm by probes having different configurations; and

FIGS. 8A-8D show experimental A-scans obtained with an excitation from SH₁ to SH₄ with 20 cycle Hann windowed signals centered respectively at 524, 588, 682 and 795 kHz in a configuration using curved magnets and coils with an inspection line positioned at 130 mm (a) and 60 mm (b) from the center of the defect as well as their respective frequency contents (c) and (d).

### DETAILED DESCRIPTION

The present disclosure is directed to methods and devices for inspection of curved ferromagnetic objects, such as but not limited to steel pipes, using ultrasonic waves. Guided waves are mechanical perturbations that propagate between two boundaries forming a waveguide such as a plate or a pipe. These can be separated into Lamb waves (symmetric and anti-symmetric), and shear horizontal (SH) guided waves. When impinging a defect or a feature parallel to the direction of polarization SH waves will not convert to Lamb modes. Unlike Lamb waves, SH waves are less sensitive to these forces when the fluid is not viscous. In addition, the cutoff frequencies-thickness product of high-order SH modes are evenly distributed along the frequency-thickness product axis, which allows the estimation of a thickness on regular intervals.

The equations Eq.(1) and Eq.(2) are used to compute the phase and group velocity of shear horizontal modes:

$${V}_{p , n} = {V}_{S} ( \frac{2 f b}{\sqrt{4 {( f b )}^{2} - {n}^{2} {V}_{S}^{2}}} ) \qquad ( 1 )$$

$${V}_{g , n} = {V}_{S} \sqrt{1 - \frac{{( n / 2 )}^{2}}{{( f b / {V}_{S} )}^{2}}} \qquad ( 2 )$$

V_{p,n} and V_{g,n} are respectively the phase and group velocity of the n-order mode, Vₛ is the bulk shear wave velocity, f is the frequency, and b is the thickness of the waveguide. The fundamental mode SH₀ can propagate at all frequency thickness products. High order modes are constrained to propagate only above a given cutoff frequency thickness product. When this value is reached, the mode's phase velocity tends towards infinity and the group velocity towards zero. This mode can no longer propagate and is then reflected or converted to a lower order mode. When a high order mode impinges an abrupt thickness reduction, it will be converted to a lower order mode, and when the thickness of the waveguide allows, it will be converted back to its original state. Conversely, if the defect is smooth most of its energy will be reflected.

In metallic materials, corrosion is a chemical weathering by an oxidizer. This implies wear of the affected surfaces, which can be likened to a local loss of thickness. For a high-order horizontal shear mode, this will shift the frequency-thickness product. If the severity of the defect is sufficient, then the mode can reach its cut-off threshold. The energy of this mode will then no longer be able to propagate. By identifying the last mode capable of propagating and the first mode filtered out, estimating the waveguide thickness is possible. Considering a plate of given thickness b, the cutoff frequency-thickness product of SHₙ can be obtained using Eq.(3):

$$f b = \frac{n {V}_{S}}{2} \qquad ( 3 )$$

Vₛ is the bulk shear wave velocity. The use of multiple of modes makes it possible to increase the number of detection thresholds. However, the excitation and detection of high order mode become more complex as the frequency increases. The attenuation may be modeled using equation Eq.(4):

I=I₀e^{-2ax} (4)

I is the intensity of the wave at a distance x from its source, I₀ is the initial intensity, and a is the attenuation coefficient depending on the material properties and increasing with the frequency. Further than attenuation, the ultrasonic wave is also subject to scattering when it encounters a defect. High-order modes can be described as dispersive. The difference between their phase and group velocities implies an alteration in the waveform in the time domain along with its propagation. These phenomena affect higher order modes more strongly. Their experimental uses over a large propagation distance are therefore more complex than for the first SH modes.

The probe described herein uses shear horizontal (SH) ultrasound waves for non-destructive testing. In some embodiments, the ultrasonic probe is used to determine a thickness profile of the curved ferromagnetic object. Generally, the probe is designed with a geometry that maximizes proximity of the probe with the object over a large surface area. In particular, a curved undersurface of the probe is used to minimize the influence of distance on a force field generated by the probe in the object. In some embodiments, the probe is custom-made to match the shape and outer-diameter of the object under inspection, thus allowing for a constant and minimal spacing between the probe and the object. Alternatively, a probe design may be used with objects having a range of outer-diameters while still benefitting from an optimized generation of Lorentz forces for a ferromagnetic object, which can alter the direction of a magnetic field due to the attraction forces between the probe and the object. The Lorentz force created by the probe is dependent on the amplitude and the direction of the magnetic field as generated and the curved nature of the undersurface of the probe helps mitigate the effect of the ferromagnetic material on the magnetic field.

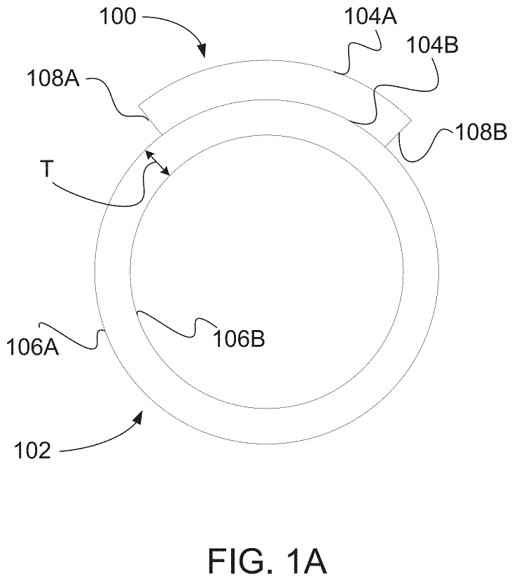

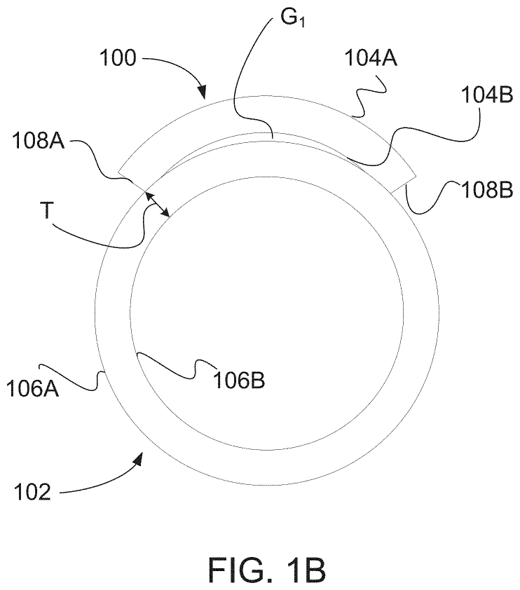

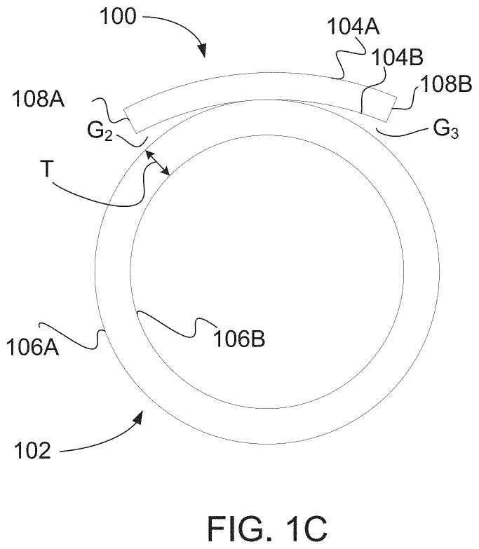

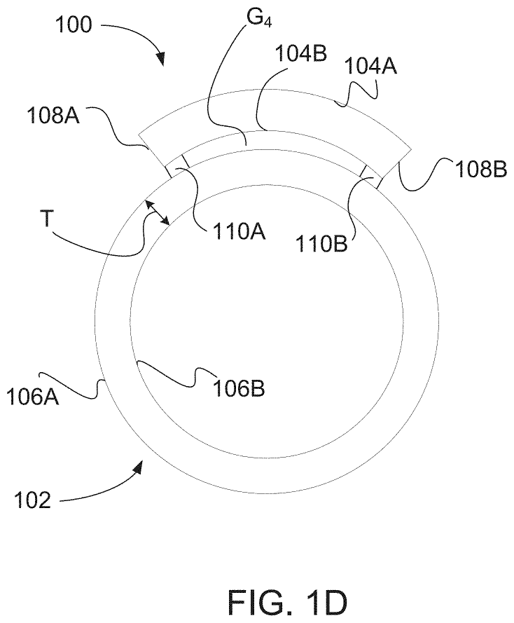

With reference to FIG. 1A, there is illustrated a cross-sectional view of an example ultrasonic probe 100 coupled to an object 102 having an inner surface 106B and an outer surface 106A. The probe 100 defines an outer surface 104A and an inner surface 104B adjacent to the outer surface 106A of the object 102. The probe 100 has an arrangement of magnets and coils that allow the generation and the detection of SH guided waves. In particular, eddy currents are created in the object 102 that lead to a Lorentz force, allowing the transduction of the SH waves longitudinally in the object 102. The inner surface 104B of the probe 100 has a non-zero curvature C₁, which is used herein to denote an amount by which the inner surface 104B deviates from a straight line. The curved inner surface 104B optimizes the generation of the Lorentz force by creating a better coupling between the probe 100 and the object 102. In some embodiments, and as illustrated in FIG. 1A, the curvature C₁ of the inner surface 104B of the probe 100 is substantially equal to a curvature C₂ of the outer surface 106A of the object 102, such that the distance between the probe 100 and the object 102 is substantially uniform across a circumferential direction of the object 102 from a first end 108A of the probe 100 to a second and opposite end 108B of the probe 100, and C₁=C₂. In this example, the entire inner surface 104B of the probe forms a region of contact with the outer surface 106A of the object 102.

In some embodiments, the curvature C₁ of the inner surface 104B of the probe 100 and the curvature C₂ of the outer surface 106A of the object 102 are different. A first example is shown in FIG. 1B, where the curvature C₁ of the inner surface 104A of the probe 100 is greater than the curvature C₂ of the outer surface 106A of the object 102, i.e. C₁>C₂. This configuration results in a gap G₁ between the inner surface 104B and the outer surface 106A, and regions of contact between the probe 100 and the object 102 proximate to and/or at the ends 108A, 108B of the probe 100. A radial dimension of the gap G₁ relative to a central axis of the object 102 decreases in a circumferential direction of the object 102 towards the opposed ends 108A, 108B of the probe. A maximum radial dimension for gap G₁ depends on many criteria, such as the properties of the material inspected, the power of the magnets, the thickness and the density of the coil, etc. The greater the gap, the less the Lorentz forces generated will be strong in emission and the harder it will be to detect the SH wave in reception.

Another example is shown in FIG. 1C, where the curvature C₁ of the inner surface 104B of the probe 100 is less than the curvature C₂ of the outer surface 106A of the object 102, i.e. C₁<C₂. Gaps G₂, G₃ are formed between the object 102 and the probe 100 at respective ends 108A, 108B of the probe 100. A radial dimension of the gaps G₂, G₃ decreases in a circumferential direction of the object 102 towards a center of the probe 100 until contact is made between the inner surface 104B of the probe and the outer surface 106A of the object at a region of contact. The size of the region of contact will depend on the difference between the curvatures C₁ and C₂. Generally, an optimal configuration is when the region of contact extends from the first opposed end 108A to the second opposed end 108B of the probe, i.e. C₁=C₂. When starting from a flat probe, the results improve as the region of contact increases, such that the curvatures of the inner surface 104B of the probe 100 and the outer surface 106A of the object 102 move towards a same value.

In some embodiments, the probe 100 is spaced apart from the object 102 by one or more support, such that contact between the probe 100 and the object 102 occurs via the one or more support. The supports are made of a material that does not impede the creation of eddy currents in the object, such as rubber, ceramic, plastic, and other non-conductive materials. An example is shown in FIG. 1D, where a first support 110A is proximate to the first end 108A and a second support 110B is proximate to the second end 108B. A gap G₄ is formed between the inner surface 104B of the probe 100 and the outer surface 106A of the object 100 between the supports 110A, 110B. In some embodiments, a radial dimension of the gap G₄ is substantially uniform in the circumferential direction. Alternatively, the radial dimension of the gap G₄ varies circumferentially.

Although the probe 100 is illustrated in the examples of FIGS. 1A-1D as having an outer surface 104A that mirrors the shape of the inner surface 104B, the outer surface 104A may be configured with any shape, including straight, concave, convex, and other geometries. A handle may be provided on the outer surface 104A for ease of manipulation by an operator. The probe 100 may be provided in a casing or housing.

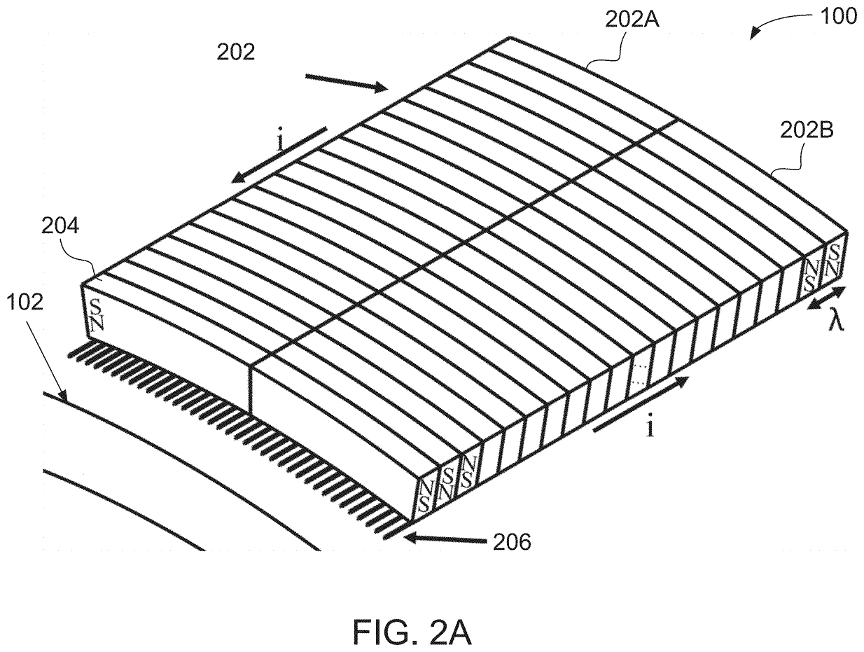

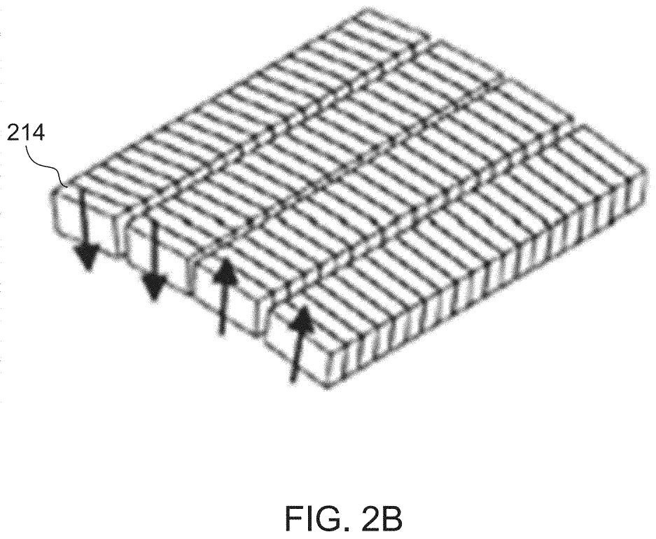

The ultrasonic probe 100 has an arrangement of magnets and coils that allow the generation and the detection of SH guided waves. With reference to FIG. 2A, an example configuration of magnets and coils is shown for the probe 100. An array 202 of magnets 204 is disposed on an elongated electrical coil 206. The probe 100 generates an ultrasonic pulse within the object 102 to which the probe 100 is coupled. The magnets 204 are disposed with periodically alternating north (N) and south (S) poles, which sets the primary wavelength of the ultrasound generated. Although the array 202 is shown with two rows 202A, 202B of magnets 204, a single row or more than two rows may be used. The electrical coil 206 runs in a direction of the alternation of the magnet poles, and when current (i) is pulsed through the coil 206, eddy currents are created in the object 102 that lead to a Lorentz force, allowing the transduction of the SH waves. More specifically, the interaction between induced eddy currents J in the conductive material of the object 102 and a magnetic field B generates Lorentz forces f_{L}:

f_{L}=J×B (5)

The proximity of the coil 206 to the conductive waveguide (i.e. the object 102) induces the eddy currents necessary for transduction. To optimize the generation of Lorentz forces, the magnet array 202 and coil 206 are curved. Since the object 102 is ferromagnetic, it has a lower reluctance than air and will tend to attract magnetic flux lines. The amplitude of the eddy currents generated by the coil 206 decreases as a function of its distance to the conductor, which has the effect of locally altering the direction of the magnetic flux lines, the amplitude of the eddy currents, and thus the shape of the Lorentz forces. The curved coil 206 minimizes the influence of the distance from the coil 206 on the generated force field. The curved array 202 ensures constant and minimal spacing of the magnets 204 and coil 206 from the object 102.

In some embodiments, curved magnets 204 are used to form the curved array 202. In one specific and non-limiting embodiment, two rows of twenty curved permanent magnets are used, as shown in FIG. 2A. Alternatively, and as shown in FIG. 2B, small rectangular magnets 214 may be used and provided in multiple rows that are relatively positioned to provide the desired curvature. This embodiment allows magnet liftoff relative to the object 102 to be reduced using only readily available magnets.

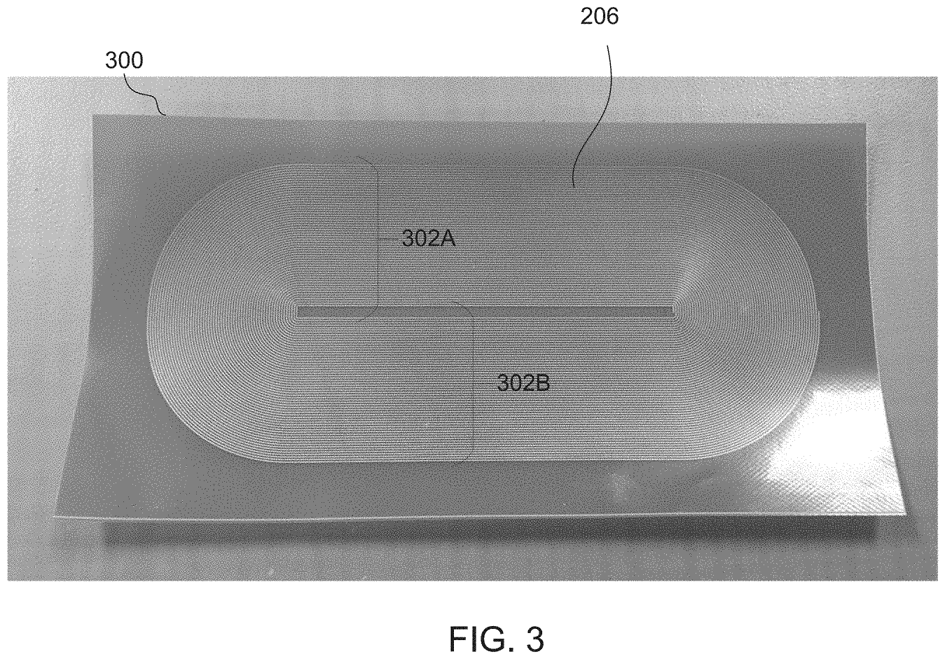

As shown in FIG. 3, the coils 206 may be provided on a flexible substrate 300, such as a flexible printed circuit board (PCB), or a rigid substrate have a pre-defined shape that corresponds to the desired shape. In this example, the coils 206 are formed as a race track, whereby a first section 302A is associated with the first row 202A of the array 202 of magnets 204 and a second section 302B is associated with the second row 202B of the array 202 of magnets 204. The configuration of the coils 206 will vary as a function of the arrangement of the magnet array 202.

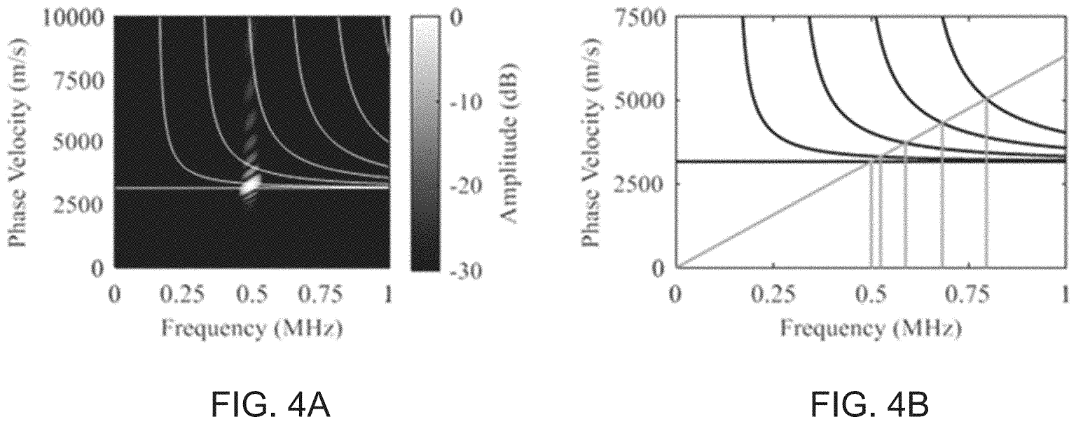

The wavenumber bandwidth of the probe 100 depends on the size and number of magnets 204, 214 in the direction of propagation. These two values make it possible to obtain a polarization pattern. By applying a Fourier transform, the amplitude of excitation as a function of the wavenumber can then be obtained. Combining this spatial spectrum with the frequency spectrum of the excitation signal allows the generation of a map of the transmitted energy and a prediction of the generated modes. A map as a function of phase velocity 17, and frequency (as shown in FIG. 4A) can be obtained using the equation Eq.(6):

$${V}_{p} = \frac{2 π f}{k} \qquad ( 6 )$$

where f is the frequency, and k is the wavenumber. By considering the excitation of a the probe 100 around a wavelength corresponding to twice the pitch of the magnets, it is then possible to predict that on the dispersion curves, the majority of the energy of the ultrasonic wave will be concentrated around a straight line inclined with a slope equal to A (as shown in FIG. 4B). The intersection of this line with the phase velocity dispersion curves of the different SH modes makes it possible to obtain the excitation frequencies of these modes. It is then possible to compute the cutoff thicknesses of the high order SH modes using Eq.(3). It is then possible to target certain modes during the emission of an ultrasonic wave.

The angle of divergence of the ultrasound beam and the near field's size may be used to estimate the capacities of an inspection. If the defect is too close to the probe 100 or too small compared to the ultrasonic beam's width, it may not have sufficient influence on the propagation of the ultrasonic wave to be detected. These two dimensions can be calculated as follows:

$$N = \frac{{D}^{2}}{4 λ} \qquad ( 7 )$$

$$sin ( θ ) = 0.44 \frac{λ}{D} \qquad ( 8 )$$

where N is the near field's length, D the dimension of the transducer in the direction perpendicular to the propagation direction, λ is the wavelength, and θ is the divergence angle from the centreline to the −6 dB line.

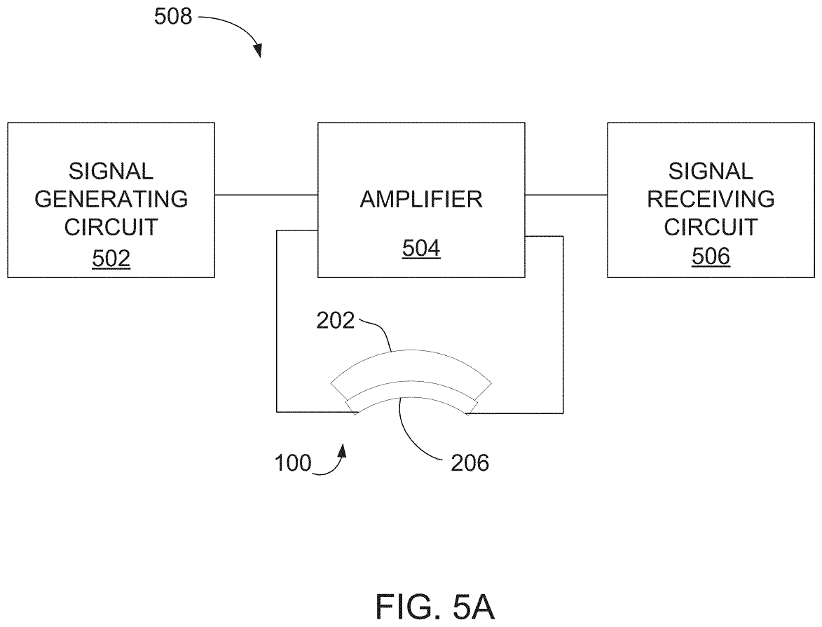

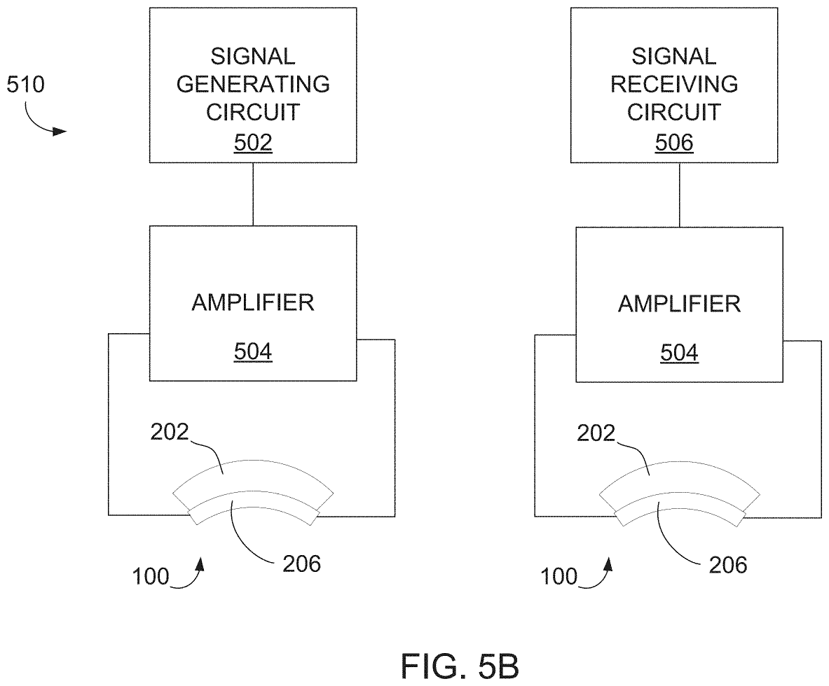

In some embodiments, a single probe 100 is used to generate and detect SH waves. An example embodiment is shown in FIG. 5A. A measurement system 508 comprises a signal generating circuit 502 that generates an emission signal and transmits the emission signal to an amplifier 504 for amplification thereof. The amplifier 504 is connected to each end of the coil 206, and the amplified signal is input into the coil 206. The amplifier 504 is also connected to a signal receiving circuit 506, such that a detected signal is received from the coil 206 into the amplifier 504 and transmitted to the signal receiving circuit 506 for processing thereof. In this configuration, transmission and detection is performed by the probe 100 sequentially. In some embodiments, two probes 100 are used, one for generation of SH waves and one for detection of SH waves. An example is shown in FIG. 5B, a measurement system 510 comprises two probes 100, a signal generating circuit 502, a signal receiving circuit 506, and two amplifiers 504. It will be understood that the circuitry used for generating and detecting the SH waves, including amplification, may be packaged separately from the probe 100, which may be identical whether used in the generation or detection of the SH waves. In this manner, a casing may house the probe 100 and provide input and output ports for connection to the various circuitry needed to operated the probe 100. Alternatively, the measurement system 508 is provided in a single housing. Also alternatively, the measurement system 510 may be provided in a first housing for generation (signal generating circuit 502, amplifier 504, probe 100) and a second housing for detection (signal receiving circuit 506, amplifier 504, probe 100).

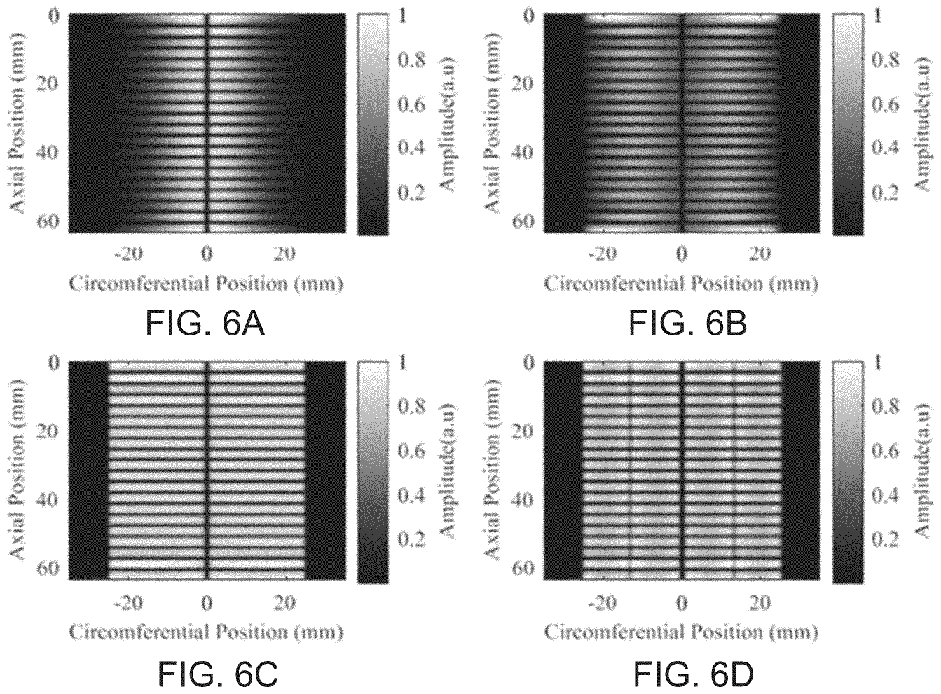

In order to demonstrate the improved performance of the probe 100 having a curved undersurface, a comparison was performed between various configurations. A reference was set using a flat probe on a flat object (i.e. a plate), with a pitch of 3.2 mm, an elevation of 50.8 mm, and an aperture of 64 mm (20 magnets in the direction of propagation) Values for near-field N and divergence angle θ of the ultrasonic beam were found to be 100.8 mm and 3.2°, respectively. The first point of comparison between the configurations is the shape of the ultrasonic field generated, which can be approximated analytically using Eq.(7) and Eq.(8). With reference to FIGS. 6A-6D, there are illustrated examples of a maximum amplitude of Lorentz Forces generated on a steel pipe with a diameter of 323.8 mm and a thickness of 10.mm by four different probe configurations. The image of FIG. 6A was obtained with a probe having rectangular magnets and a flat coil on the steel pipe. The effective elevation of the probe is 24.3 mm. This large deviation from the reference value is due to the increasing liftoff of the coil and magnets at the edge of the probe, due to the flatness of the undersurface. FIG. 6B was obtained with a probe having rectangular magnets and a curved coil. The elevation increases to 41.7 mm due to a uniform spacing of the curved coil with the pipe. FIG. 6C was obtained with curved magnets and a curved coil, as illustrated in the example of FIG. 2A, and FIG. 6D was obtained with small rectangular magnets and a curved coil, as illustrated in the example of FIG. 2B. In both cases, an elevation close to the reference value is obtained, respectively at 50.7 and 50.6 mm. This variation in the effective elevation of the probe, according to Eq.(7) and Eq.(8), will induce an alteration in the shape of the generated ultrasonic field.

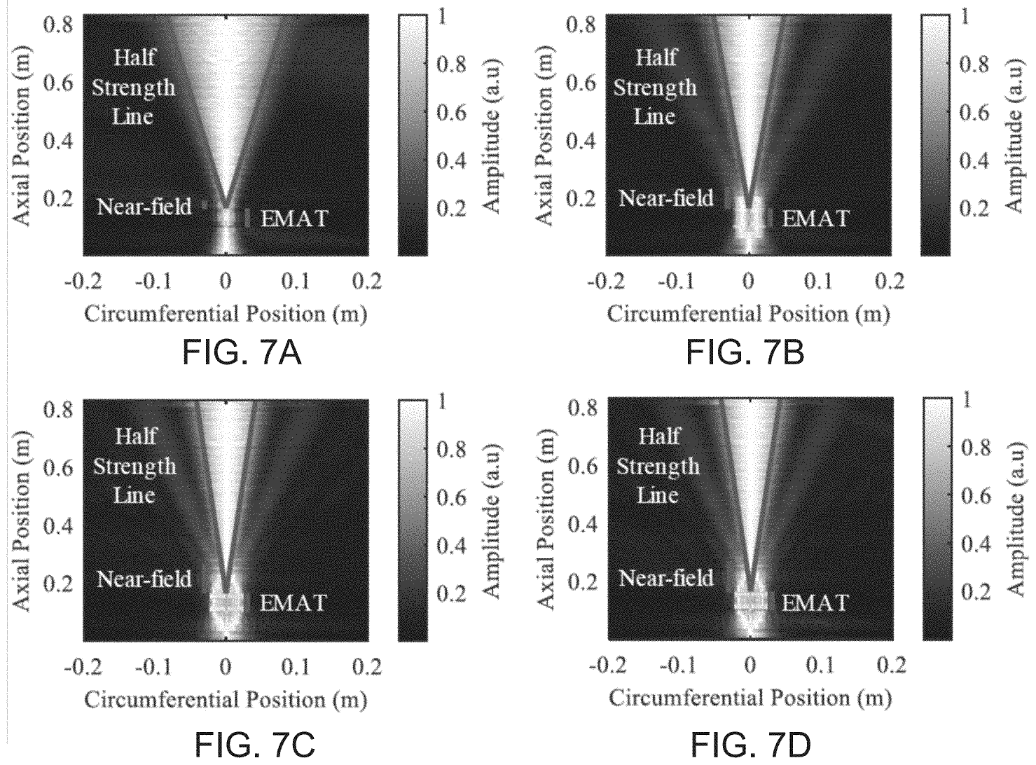

A reduction of the elevation reduces the size of the near field of the probe and increases the angle of divergence of the ultrasound beam. Diffraction patterns of the different probe configurations are shown in FIGS. 7A-7D. FIG. 7A corresponds to the diffraction pattern for the probe configuration having a flat coil and rectangular magnets on the steel pipe. This case represents the most divergent configuration since the angle obtained is 106% greater than the reference value obtained for an elevation of 50.8 mm. By using a flexible coil that best follows the curvature of the pipe (FIG. 7B: rectangular magnets and a curved coil) this effect is reduced since an increase of 19% is noted. Finally, the cases with a curved magnet array and coil (FIG. 7C: curved magnets and a curved coil; 7D: small rectangular magnets and a curved coil), present results similar to the reference case.

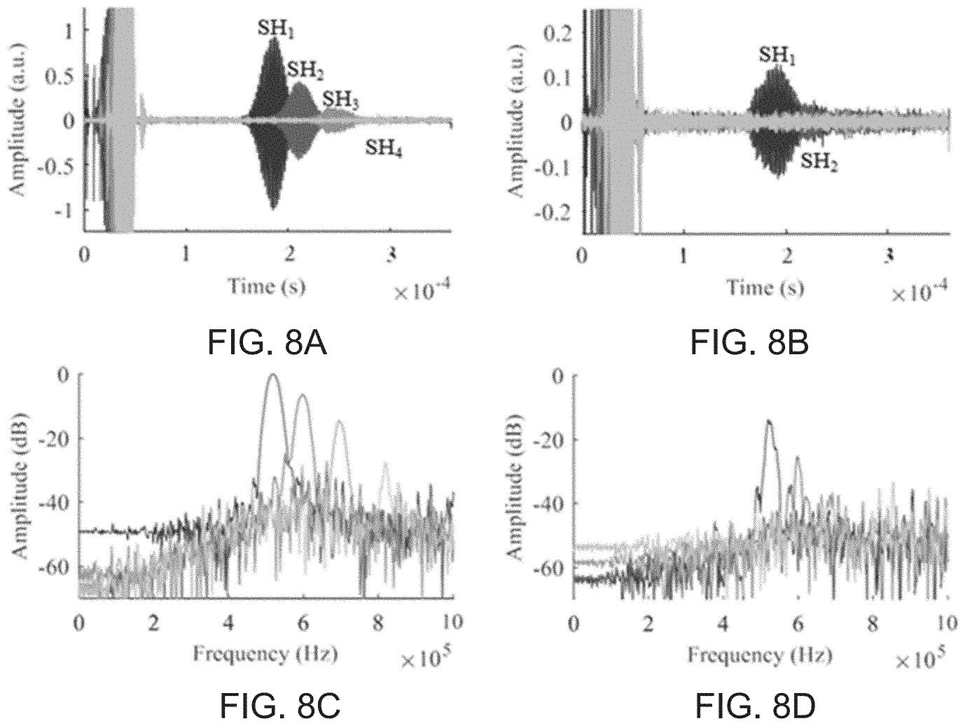

As shown in FIGS. 6A-6D and 7A-7D, the least divergent cases are for probe configurations having a curved magnet array and coil. FIGS. 8A-8C show two scans performed at two different positions along the circumference of a pipe with probes made up of curved magnets. FIGS. 8A and 8C correspond to a reference measurement without defect on the inspection line. More precisely, these measurements were taken 130 mm from the center of the defect on the circumferential axis. At this position, the thickness on the inspection line corresponds to the nominal thickness of the pipe. By analyzing the frequency content of the various A-Scans, it can be seen that all the modes from SH₁ to SH₄ can propagate. It should be noted that the amplitude of SH₄ is much lower than that of the other modes. It is therefore deduced that the thickness of the waveguide must be between the nominal thickness of the pipe (10.2 mm) and the cut-off thickness of SH₄ (8 mm). In FIGS. 8B and 8D, the inspection line passes through the defect, and the minimum thickness is 6 mm. Here we see a significant reduction in the amplitude of the reception signals, which is characteristic of the presence of a defect. It is nevertheless possible to note the presence of SH₁ and SH₂ in the frequency content of the signals. SH₃ and SH₄ were not detected. The thickness must therefore be between the cut-off thickness of SH₃ (7 mm) and that of SH₂ (5.4 mm).

The comparative study of the four probe configurations and their ability to reconstruct the thickness profile of a steel pipe has demonstrated that the lift-off distance between magnets and the coil from the pipe can have a significant effect on the generation of the ultrasonic wave. Experimentally, this has manifested itself as a significant loss of signal-to-noise ratio which can complicate the excitation or detection of high-order SH modes. The probe having a curved undersurface allowed the pipe thickness profile to be reconstructed using the cutoffs from SH₂ to SH₄. This technique, therefore, makes it possible to detect at most a loss of 50% of the thickness of the waveguide.

As can be seen therefore, the examples described above and illustrated are intended to be exemplary only. The scope is indicated by the appended claims.

1. A probe for non-destructive testing of a curved object, the probe comprising an arrangement of magnets and coils configured for generating shear horizontal guided waves for propagating longitudinally in the object, the probe having a top surface, a bottom surface, and two opposed ends extending between the top surface and the bottom surface, the bottom surface having a non-zero curvature between the two opposed ends and matable with an outer surface of the curved object.

2. The probe of claim 1, wherein the arrangement of magnets and coils comprises a curved array of magnets disposed on an elongated electrical coil residing on a curved substrate.

3. The probe of claim 2, wherein the curved array of magnets comprises at least one row of curved magnets disposed with periodically alternating north and south poles.

4. The probe of claim 3, wherein the at least one row of curved magnets comprises two rows of curved, permanent magnets.

5. The probe of claim 2, wherein the curved array of magnets comprises a plurality of rows of rectilinear magnets positioned along a curved path.

6. The probe of claim 1, wherein the non-zero curvature of the bottom surface of the probe matches a curvature of the outer surface of the curved object.

7. The probe of claim 1, wherein the non-zero curvature of the bottom surface of the probe is greater than a curvature of the outer surface of the curved object.

8. The probe of claim 1, wherein the non-zero curvature of the bottom surface of the probe is less than a curvature of the outer surface of the curved object.

9. The probe of claim 1, wherein the magnets are permanent magnets.

10. The probe of claim 1, wherein the coils are mounted to a flexible substrate.

11. The probe of claim 1, wherein the coils are mounted to a rigid substrate.

12. The probe of claim 1, wherein the bottom surface of the prove is matable with the outer surface of the curved object via at least one support.

13. A measurement system comprising:
at least one probe according to any one of claims 1 to 12;
a signal generating circuit for generating an emission signal; and
a signal receiving circuit for receiving a detection signal.

14. The measurement system of claim 13, further comprising at least one amplifier connected to ends of the coils of the at least one probe, to the signal generating circuit, and to the signal receiving circuit, the signal generating circuit configured for transmitting the emission signal to the at least one amplifier for amplification thereof.

15. The measurement system of claim 14, wherein the at least one amplifier is configured for receiving the emission signal from the signal generating circuit, for generating an amplified signal based on the emission signal, and for inputting the amplified signal into the coils.

16. The measurement system of claim 15, wherein the at least one amplifier is configured for receiving the detection signal from the coils, and for transmitting the detection signal to the signal receiving circuit.

17. The measurement system of claim 13, wherein the at least one probe comprises a single probe configured for sequentially generating and detecting the shear horizontal guided waves.

18. The measurement system of claim 13, wherein the at least one probe comprises a first probe configured for generating the shear horizontal guided waves and a second probe configured for detecting the shear horizontal guided waves.
